(19) United States
(12) Patent Application Publication
(10) Pub. No.: US 2003/0150137 A1
(43) Pub. Date: Aug. 14, 2003

(54) Orthopedically adjustable shoe construction

(76) Inventor: Henri E. Rosen, Watertown, MA (US)

Correspondence Address: Jacobs Patent Office, P.O. Box 390438, Cambridge, MA, 02139, US

(21) Appl. No.: 10/073,803

(22) Filed: Feb. 11, 2002

(51) Int. Cl. A61F 5/14; A43B 7/24

(52) U.S. Cl. 36/144; 36/142

(57) ABSTRACT

A shoe construction is described in which generally concealed means can be manually adjusted to change the degree of transverse orthopedic foot support provided by the shoe.

### BACKGROUND OF THE INVENTION

In the past, there had long been a need for a shoe construction offering practical means of adjustment of the degree of orthopedic support provided by the shoe by the wearer thereof. This inventor's previous patents and in particular U.S. Pat. No. 6,226,901 met this need, but with some inherent limitations in foot comfort leading to the significant improvements in the overall approach which are the subject of the present invention.

Accordingly, it is an object of this invention to provide a shoe construction comprising improved concealed canting adjustment means whereby the wearer of the shoe can manually adjust the degree of canting to produce more comfortable orthopedic support. It is a further object to make the concealed canting adjustment means preferably non-removable from the shoe. It is a still further object of the invention to provide the canting adjustment means in a manner such that the entry of foreign matter such as sand, pebbles and the like within the adjustment means is precluded.

For a fuller understanding of the nature and objects of the present invention, reference should be made to the following detailed description taken in connection with the accompanying drawings.

### DISCLOSURE OF THE INVENTION

The present invention is directed to a shoe construction comprising concealed and integral wearer-adjustable orthopedic support means to provide an adjustable degree of variable transverse angular canting support to a weight-bearing foot thereon. The adjustment means comprises at least one transversely adjustable side element in the form of appropriately contoured wedge assemblies manually movable by a screw-operated cam. Attached to an insole base element and variably supporting a contoured plastic foot support immediately thereabove, with the assembly thereof protected against the unwanted infiltration of foreign matter such as dirt and pebbles by a fabric covering element. The canting adjustment means provides a variably adjustable degree of canting support to a weight-bearing foot thereupon.

### BRIEF DESCRIPTION OF THE DRAWINGS

FIG. 1 is a side view of a shoe construction embodying principles of the present invention.

FIG. 2 is a plan view of the insole assembly of the shoe of FIG. 1 taken along line 2-2 thereof and showing the canting means when set for zero degrees of cant, i.e. horizontal in attitude.

FIG. 3 is a plan view of the insole assembly of the shoe of FIG. 1 taken along line 3-3 thereof and showing the canting means when adjusted to an effective angle of four degrees (‘positive’) cant relative to the horizontal.

FIG. 4 shows a transverse cross-sectional view of the insole assembly of FIG. 3 taken along line 4-4 thereof.

FIG. 5 shows a transverse cross-sectional view of the insole assembly of FIG. 3 taken along line 5-5 thereof.

### DETAILED DESCRIPTION OF THE INVENTION

Improved means for the manual adjustment of the effective angular predominantly transverse tilting or ‘cant’ of a shoe footbed assembly will be described with reference to a so-adjusted support system preferably integral to the shoe and non-removable therefrom. It should be understood that similar means could be designed to be removably insertable relative to the shoe, and such means is to be considered equivalent to the means of the invention but not a preferred embodiment as such removable assemblies would tend to be used in footwear not designed therefor, usually resulting in adverse fit and function therewith.

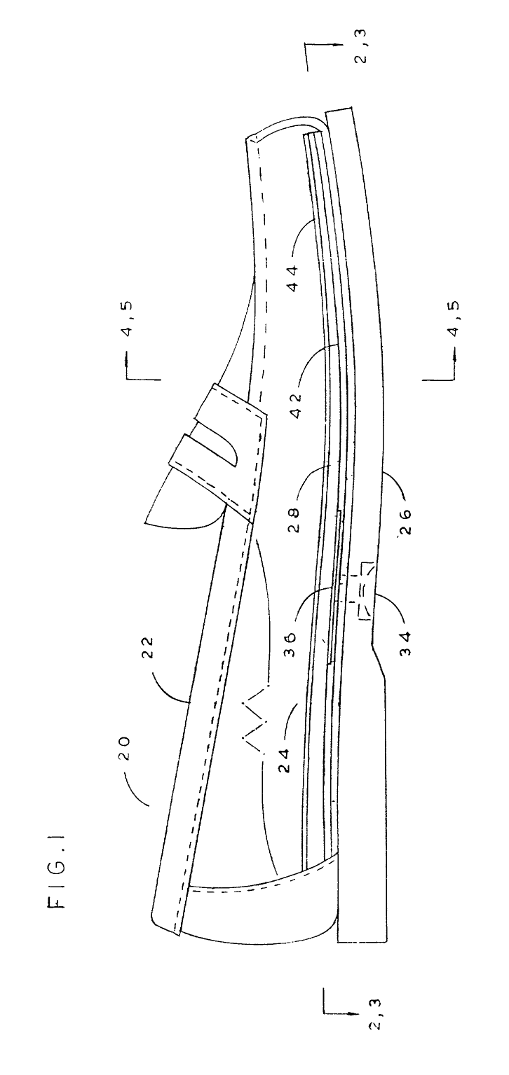

Referring to the drawings, FIGS. 1 through 5 show embodiments of the adjustable foot support system of the present invention as it would appear used in the popular saddle loafer casual shoe style. It will be understood that this system and approach will be readily applicable to most other shoe designs and categories as well. It will obviously also be applicable to sandals, boots, skates and other athletic and non-athletic applications as well.

FIG. 1 shows a shoe containing the shoe construction of this invention. More specifically, it shows a shoe 20 having an upper 22, an adjustable footbed canting assembly 24 and a sole 26. The footbed canting assembly 24 comprises a preferably molded insole element 28 and at least one transversely adjustable side wedge 30. As shown side wedge 30a is along the inner side of a wearer's foot and side wedge 30b is on the outer side of the wearer's foot. Preferably, the two side wedges 30a and 30b are interconnected by integral hinge portions 32 at the toe and heel so that the side wedges will move together to change the cant of the top surface of the footbed canting assembly 24. The transverse positioning of the side wedges 30 adjusts the effective degree of the transverse canting attitude of the top surface of footbed canting assembly 24.

Although less preferable, a single side wedge 30 may be used. For example side wedge 30a along the inner side of a wearer's foot and having a hinge 32 in its central portion facing outward may be used alone to control pronation and side wedge 30b on the outer side of the shoe and having a hinge 32 facing inward can be used alone to control supination

The positioning of the side wedges 30a and/or 30b is controlled by a manual adjustment means 36 located under insole foot supporting element 28 in a mid-portion of the shoe 20. Locating the adjustment means 36 forward of the breast of the heel serves to minimize abrasion on the adjustment means during use. The side wedges 30a and 30b in turn support variably with such adjustment controlled by the coin-slotted adjusting screw 34 attached as by spot-welding to a stainless steel circular stamped cam 36 which is attached to wedges 30 by eyelets 38 extending through the variably radiused arcuate cam slots 40 in cam 36 and through optional transverse slots in the insole base 42 thereunder. Preferably the footbed canting assembly 24 is enclosed by a spandex fabric or other sheet covering element 44, permanently attached as by cement lasting to the bottom peripheral edges of insole base 42, with the edge surface areas of the footbed canting assembly 24 similarly are permanently attached to the marginal so-called lasting allowances as well as the assembly forepart of upper 22 by such permanent attaching means as adhesive cement.

Optionally, the footbed canting assembly 24 may be designed without the insole element 28 by using one of wedge assemblies 30a and 30b alone to support the foot under covering 44. This embodiment is not preferred as it is unlikely that such wedges could be designed to give optimum foot comfort and edge support while providing an adequate range of canting adjustment therewith.

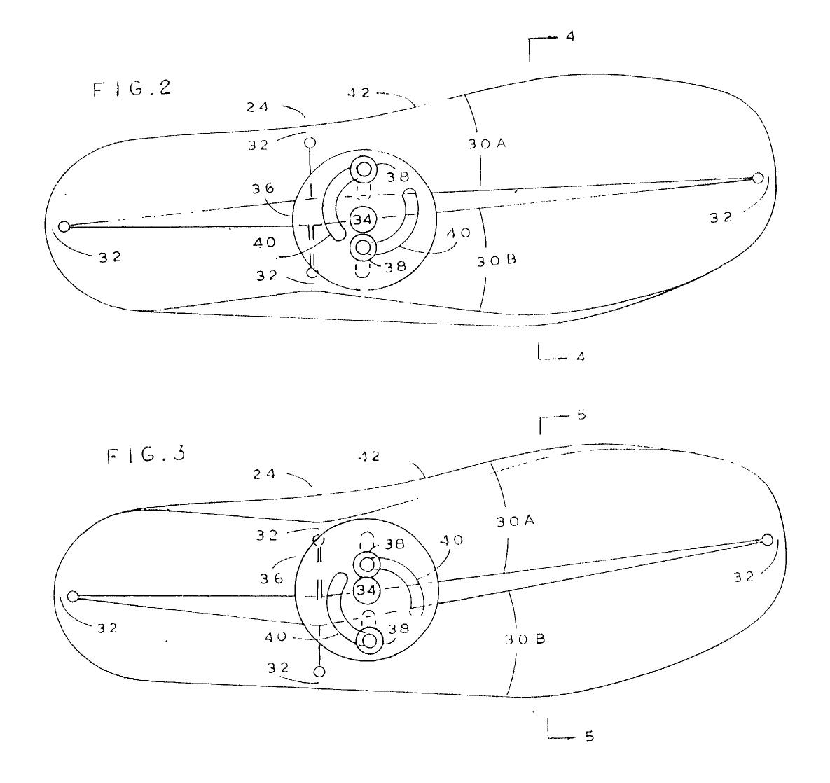

FIG. 2 shows a simplified plan view of the above elements of the canting adjustment means of assembly 24 as it would appear when horizontal, i.e. at zero degrees of effective transverse cant.

FIG. 3 shows a similar plan view of said means as it would appear adjusted to a maximum effective canting angle of four degrees ‘positive,’ as this angular attitude is referred to when adjusted to correct for a maximum degree of pronation, the excessive inward rolling of the foot and ankle relative to the longitudinal axis of the foot (and shoe).

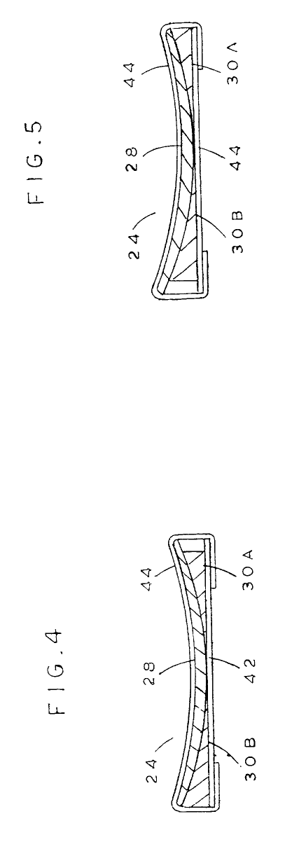

FIG. 4 shows a transverse cross-sectional view of the footbed assembly 24 of FIG. 1 showing the insole element 28 in an effectively horizontal attitude of zero degrees canting adjustment as in the adjustment of FIG. 2.

FIG. 5 shows the transverse cross-sectional view of the footbed assembly 24 of FIG. 1 showing a four degree positive effective canting attitude corresponding to the adjustment of the means, shown in FIG. 3.

It will be noted that the preferred canting adjustment movement of the side wedge elements 30a and 30b is predominantly transverse to the longitudinal axes of the canting assembly 24 and shoe 20. As shown, the canting adjustment is infinitely variable over the range of from zero to about four degrees positive, with the latter being the currently preferred maximum correction at heel and/or ball for a foot experiencing excessive pronation, these areas being so corrected as they tend to share most of the weight-bearing function of the foot through the stride cycle.

Optionally, the correction adjustment can be altered as to degree, range, negative and/or positive correction and correction location, by the appropriate design of the assembly to the preference of a manufacturer and marketer of this system. As noted, similar canting correction is available for correction of excessive supination, the excessive outward rolling of the foot and ankle that is the opposite condition to that of pronation, but far less frequent in occurrence. While correction for this condition could be offered in a system that also corrected for pronation, it is considered preferable at this point to offer such correction separately, to prevent trauma that could result from errors in adjustment by wearers, the majority of whom need correction solely for pronation.

As to materials for the shoe construction of this invention, the upper and sole may be of any of the wide range of materials conventionally used in footwear, including natural and synthetic leather, fabrics, plastics and combinations thereof. The elements of the footbed canting assembly 24 can be from a wide range of materials, those preferred include: a non-woven socklining fabric for covering 44, such as Cambrelle® from Faytex Corp. of Braintree, Mass. Insole 28 and side wedges 30 can be of a polyolefin such as polyethylene, molded by suppliers as Applied Plastic Tech. of Worcester, Mass. Insole base 42 can be of DuPont's Hytrel®, extruded together with a fabric base layer by Bixby Int'l. of Newburyport, Mass. Circular cam 36 will be supplied, welded to stainless steel screw 34 by Peter Forg, Inc., of Somerville, Mass. Shoulder eyelet/washer assemblies 38 will be from Stimpson, Inc., of Bayport, N.Y.

While the construction disclosed cites preferred embodiments of the invention, it should be understood that alternative options would include other ramp arrangements and motions as well as screw and/or lever means similarly supporting the foot while preventing entry of foreign matter into the adjustment means, all less preferred due to complexity and/or necessary rigidity of such means. Other equivalents would include means for manual adjustment from within the shoe as by a cam-adjusting screw located at the top of the footbed canting assembly 24, directly under a conventional removable socklining in a similar location to that of the drawing, for example.

While the canting footbed assembly 24 shown in the drawings is full shoe length, shorter assemblies are included herein. For example a three-quarter length backpart footbed assembly may be preferred for either casual styles such as a loafer or for those dress shoe styles where a minimum of forepart vertical height of the finished shoe is desired. Since the three-quarter or similar length backpart footbed assembly precludes forepart canting, it is not currently preferred.

It should be understood that the above disclosure represents only a few applications of the concepts of this invention and that other arrangements of wedges, inserts, levers and/or screw means that function in the same or similar fashion are considered possible and equivalents to the invention under its teachings.

What is claimed is:

1. A shoe comprising a shoe having an upper, a sole, and an adjustable footbed canting assembly comprising an adjustably movable side wedge which moves predominantly laterally relative to the length-wise axis of the shoe to change the transverse canting attitude of the footbed canting assembly relative to a weight-bearing foot thereover and supported thereon.

2. The shoe of claim 1, further containing means to prevent entry of unwanted foreign matter.

3. The shoe of claim 1, wherein the adjustable support means is not removable from the shoe.

4. The shoe of claim 1, wherein the adjustable movable side wedge is moved by a manually operable means.

5. The shoe of claim 4, wherein the manually operable means comprises a manually adjusted cam located in a mid-portion of the shoe forward of the breast of the heel.

6. The shoe of claim 4, wherein the manually operable means are controlled from an outer surface of the shoe.

7. The shoe of claim 4, wherein the manually operable means are controlled from an inner surface of the shoe.

8. The shoe of claim 1, wherein the adjustable footbed canting assembly is removably insertable in the shoe.

9. An adjustable foot support system for use in a shoe, having a toe portion, a heel portion, and a mid-portion therebetween, comprising
(i) a footbed assembly which has a longitudinal center, a flat bottom surface and an upper surface which has raised peripheral edges at the mid-portion that slope gradually downward from each edge toward the longitudinal center of the foot-bed assembly so as to form a concave shaped surface facing upward at about the mid-portion;
(ii) a shim member having a toe portion, a heel portion, a mid-portion there-between and comprising a wedge, wherein the wedge is joined at the toe portion and the heel portion and has a central hinge area therebetween, and having a flat upper surface and a lower surface which has a transversely wedged contour at about the mid-portions of the shim facing downward, thereby matching and fitting together with the footbed assembly located therebelow, and having the shim mid-portion being narrower than the corresponding footbed assembly mid-portion location, and
(iii) a means located forward of the breast of the heel for moving the wedge transversely to adjust the angle of the flat upper surface of the shim member relative to the flat bottom surface of the footbed assembly.

10. The adjustable foot support system of claim 9, wherein the wedge comprises a left wedge and a right wedge, wherein the left wedge and the right wedge are joined at the toe portion and the heel portion and have an unconnected central area therebetween, and wherein the left wedge and the right wedge each have a hinge extending into the unconnected central area, and have a flat upper surface and a lower surface which have a transversely wedged contour at about the mid-portions of the shim facing downward, thereby matching and fitting together with the footbed assembly located therebelow and a means located forward of the breast of the heel of the shoe for moving both the left wedge and the right wedge transversely to adjust the angle of the flat upper surface of the shim member relative to the flat bottom surface of the footbed assembly.

11. The adjustable support system of claim 10, wherein the means for moving the wedges comprises a manually adjustable cam member attached to the footbed assembly and the wedges in a manner so as to allow movement of the wedges relative to the footbed assembly.

12. The adjustable support system of claim 10, wherein the hinge in the left wedge is a slot in the mid-portion thereof extending toward the right wedge and the hinge in the right wedge is a slot in the mid-portion thereof extending toward the left wedge.

13. The adjustable support system of claim 10, wherein the system is removably insertable in a shoe.

14. The adjustable support system of claim 10, wherein the shim is substantially the same length as the footbed assembly.

15. The adjustable support system of claim 10, wherein the shim is shorter than the footbed assembly.

16. The adjustable support system of claim 10, wherein the left wedge, the right wedge, and the unconnected central area therebetween in total are about 5 to about 35% narrower than the corresponding mid-portion location of the footbed assembly.

17. A shoe comprising a shoe upper, a shoe bottom, and an adjustable foot support system in the shoe, said support system comprising: (i) a footbed assembly having a longitudinal center, a toe portion, a heel portion, and a mid-portion there-between, and having a bottom surface and an upper surface which has raised peripheral edges at the mid-portion that slope gradually downward from each edge toward the longitudinal center of the footbed assembly so as to form a concave shaped surface facing upward at the mid-portion; (ii) a shim member having a toe portion, a heel portion, a mid-portion there-between and comprising a wedge, wherein the wedge is joined at the toe portion and the heel portion and has a central hinge area therebetween, and having a flat upper surface and a lower surface which has a transversely wedged contour at about the mid-portions of the shim facing downward, thereby matching and fitting together with the footbed assembly located therebelow, and having the shim mid-portion being narrower than the corresponding footbed assembly mid-portion location, and (iii) a means located forward of the breast of the heel of the shoe for moving the wedge transversely to adjust the angle of the flat upper surface of the shim member relative to the flat bottom surface of the footbed assembly.

18. The shoe of claim 17, wherein the wedge comprises a left wedge and a right wedge, wherein the left wedge and the right wedge are joined at the toe portion and the heel portion and have an unconnected central area therebetween, and wherein the left wedge and the right wedge each have a hinge extending into the unconnected central area, and have a flat upper surface and a lower surface which have a transversely wedged contour at about the mid-portions of the shim facing downward, thereby matching and fitting together with the footbed assembly located therebelow and a means located forward of the breast of the heel of the shoe for moving both the left wedge and the right wedge transversely to adjust the angle of the flat upper surface of the shim member relative to the flat bottom surface of the footbed assembly.

19. The shoe of claim 18, wherein the means for moving the wedges extends through the bottom of the shoe.

20. The shoe of claim 18, wherein the means for moving the wedges is accessible from within the shoe.

21. The shoe of claim 18, wherein the means for moving the wedges comprises a manually adjustable cam member attached to the footbed assembly and the shim member in a manner so as to allow movement of the shim member relative to the footbed assembly.

22. The shoe of claim 18, wherein the hinge in the left wedge is a slot in the mid-portion thereof extending toward the right wedge and the hinge in the right wedge is a slot in the mid-portion thereof extending toward the left wedge.

23. The shoe of claim 18, wherein the left wedge, the right wedge, and the unconnected central area therebetween in total are about 5 to about 35% narrower than the corresponding mid-portion location of the footbed assembly.
